(19) United States
(12) Patent Application Publication
(10) Pub. No.: US 2007/0208183 A1
(43) Pub. Date: Sep. 6, 2007

(54) HYDROGENATION PROCESS FOR THE CONVERSION OF A CARBOXYLIC ACID OR AN ESTER HAVING A CARBONYL GROUP

(76) Inventors: Rene Johan Haan, Amsterdam (NL); Jean-Paul Lange, Amsterdam (NL); Leonardus Petrus, Amsterdam (NL); Catharina Johanna Maria Petrus-Hoogenbosch, Amsterdam (NL)

Correspondence Address: SHELL OIL COMPANY, P O BOX 2463, HOUSTON, TX, 772522463, US

(21) Appl. No.: 11/680,437

(22) Filed: Feb. 28, 2007

(30) Foreign Application Priority Data
Mar. 2, 2006 (EP) 06110609.2

(51) Int. Cl. C07D 305/12; C07C 69/66

(52) U.S. Cl. 549/303; 560/179; 549/328

(57) ABSTRACT

A hydrogenation process for the conversion of a reactant selected from the group consisting of a carboxylic acid having a carbonyl group and an ester of a carboxylic acid having a carbonyl group to form a carboxylic acid or an ester with a hydroxyl group or a lactone, wherein the reactant is contacted with a heterogeneous catalyst comprising a hydrogenating compound, in the presence of formic acid, at a temperature in the range of from 150 to 400° C. and a pressure in the range of from 1.0 to 150 bar (absolute).

### CROSS-REFERENCE TO RELATED APPLICATION

This application claims priority from European Patent Application No. 06110609.2, filed on Mar. 2, 2006, which is incorporated herein by reference.

### FIELD OF THE INVENTION

The present invention provides a hydrogenation process for the conversion of a reactant selected from the group consisting of a carboxylic acid having a carbonyl group and an ester of a carboxylic acid having a carbonyl group to form a carboxylic acid or an ester with a hydroxyl group or a lactone.

### BACKGROUND OF THE INVENTION

It is known that carboxylic acids having a gamma-carbonyl group, such as for example levulinic acid, or esters of such carboxylic acids can easily be converted into a gamma lactone by catalytic hydrogenation. The conversion may proceed via hydrogenation to gamma-hydroxy carboxylic acid or ester followed by intramolecular (trans)esterification to the gamma lactone or via intramolecular (trans)esterification of the enol form of the carboxylic acid or ester followed by hydrogenation to the gamma lactone. Usually such catalytic hydrogenation is carried out over a heterogeneous catalyst with a hydrogenating function, in the presence of molecular hydrogen. Catalytic hydrogenation processes for the conversion of levulinic acid into gamma valerolactone, also known as 5-methylbutyrolactone or 5-methyl-dihydro-furan-2-one, are for example disclosed in U.S. Pat. No. 5,883,266, WO 02/074760 and WO 98/26869. A process for the catalytic hydrogenation of levulinate esters to form gamma valerolactone is disclosed in EP 069 409 A1. Gamma valerolactone may suitably be used as bio-derived solvent or as intermediate for other chemical compounds.

Levulinic acid can be obtained by hydrolysis of cellulose-containing biomass. Formic acid is formed as a by-product of such hydrolysis process. A process for the manufacture of levulinic acid from biomass is for example disclosed in U.S. Pat. No. 4,897,497.

It would be advantageous if the formic acid that is obtained as by-product of the manufacture of levulinic acid could be used as hydrogen donor for the hydrogenation of levulinic acid or its derivatives into gamma valerolactone or other products.

The use of formic acid as hydrogen donor is known. In WO 01/23088 for example is disclosed a process for asymmetrical transfer hydrogenation, wherein a prochiral compound is hydrogenated with formic acid as hydrogen donor, using a homogeneous catalyst having a nitrogen-containing enantiomerically enriched ligand.

In EP 916 637 A1 is disclosed the use of a transition metal complex with an optically active nitrogen-containing compound as an asymmetric ligand, typically an optically active ruthenium-diamine complex, as catalyst for the hydrogenation of ketones using formic acid as hydrogen donor. The catalysts used in EP 916 637 A1 are homogeneous catalysts.

In Green Chemistry 4(2002)179-180, T. N. Danks and B. Desai describe a microwave assisted catalytic transfer hydrogenation using alumina-supported formate as hydrogen donor and Wilkinson's catalyst. Wilkinson's catalyst is rhodium tris(triphenylphosphine)chloride, i.e. a homogeneous catalyst.

In GB 1 575 808 is disclosed the use of formic acid as hydrogen donor for the hydrogenation of organic nitro, nitroso or hydroxylamino compounds. A noble metal supported on charcoal is used as catalyst. The reaction is carried out in liquid phase at temperatures below 100° C. Catalysts comprising a large amount, i.e. 5 or 10%, of noble metal are used.

### SUMMARY OF THE INVENTION

It has now been found that formic acid can be used as a hydrogen donor for the catalytic conversion of a carboxylic acid or an ester with a carbonyl group into a carboxylic acid or an ester with a hydroxyl group, or, in case the carbonyl group is a gamma- or delta-carbonyl group, into a gamma- or delta-lactone by using a heterogeneous catalyst with a hydrogenating function at a temperature in the range of from 150 to 400° C.

Accordingly, the present invention provides a hydrogenation process for the conversion of a reactant selected from the group consisting of a carboxylic acid having a carbonyl group and an ester of a carboxylic acid having a carbonyl group to form a carboxylic acid or an ester with a hydroxyl group or a lactone, wherein the reactant is contacted with a heterogeneous catalyst comprising a hydrogenating compound, in the presence of formic acid, at a temperature in the range of from 150 to 400° C. and a pressure in the range of from 1.0 to 150 bar (absolute).

### DETAILED DESCRIPTION OF THE INVENTION

In the hydrogenation process according to the invention, a carboxylic acid having a carbonyl group or an ester of such carboxylic acid is contacted with a heterogeneous catalyst comprising a hydrogenating metal in the presence of formic acid as hydrogen donor to hydrogenate the carbonyl group to a hydroxyl group. If the reactant is a carboxylic acid or an ester with a carbonyl group on the carbon atom that is in the gamma- or delta-position with respect to the carboxyl or ester group, the corresponding lactone, i.e. a gamma- or delta-lactone, will typically be formed by an intramolecular esterification or transesterification reaction.

The process preferably is a process for hydrogenating reactants with a gamma- or delta carbonyl group wherein a gamma- or delta-lactone is formed. More preferably, the reactant is a carboxylic acid or an ester with a gamma-carbonyl group. Examples of suitable reactants with a gamma-carbonyl group are levulinic acid, esters of levulinic acid, a dimer of levulinic acid or a mono- or di-ester of such dimer. Examples of dimers of levulinic acid with a gamma carbonyl group are 4-methyl-6-oxononanedioic acid, 3-acetyl-4-methylheptanedioic acid, or their lactones, i.e. 5-(2-methyl-5-oxotetrahydrofuran-2-yl)-4-oxopentanoic acid or 3-(2-methyl-5-oxotetrahydrofuran-2-yl)-4-oxopentanoic acid. The levulinic acid dimers mentioned above may be obtained by contacting levulinic acid in the presence of hydrogen with a strongly acidic catalyst having a hydrogenating function, e.g. Pd/cation-exchange resin, at elevated temperature and preferably at elevated pressure. Typical process temperatures and pressures are in the range of from 60 to 170° C. and of from 1 to 200 bar (absolute), respectively. Such process for levulinic acid dimerisation is described in detail in co-pending patent application PCT/EP2005/056206. The catalyst and process conditions of this process are similar to those applied in the known single-step process for the production of methyl isobutyl ketone from acetone. Such single-step methyl isobutyl ketone process is for example disclosed in Kirk-Othmer's Encyclopedia of Chemical Technology, 3rd ed., 1981, Vol. 13, p. 909, in Ullmann's Encyclopedia of Industrial Chemistry, 5th ed., 1990, Vol. A15, p. 80, and in WO 99/65851.

Levulinic acid or esters of levulinic acid are particularly preferred reactants.

With an ester as carbonyl reactant, a higher yield of the hydrogenated product, i.e. hydroxyl compound or lactone, is obtained than in case of an acid as carbonyl reactant. Therefore, the reactant preferably is an ester of a carboxylic acid with a carbonyl group, more preferably an alkyl ester. Even more preferably an alkyl ester with at most 10 carbon atoms in the alkyl group. Methyl, ethyl, butyl and pentyl esters of a carboxylic acid with a carbonyl group are particularly suitable as reactant, more in particular methyl, ethyl, butyl and pentyl esters of levulinic acid.

Formic acid and the carbonyl reactant may be in the gas phase or in the liquid phase when contacted with the catalyst. In case of a high-boiling carbonyl reactant, for example a levulinic acid dimer, formic acid may be in gas phase whilst the carbonyl reactant is in the liquid phase. Preferably, the reactants are in the gas phase.

The carbonyl reactant and formic acid may be supplied to the catalyst continuously or batch-wise. They may be supplied in undiluted form or with an inert gaseous or liquid diluent.

The molar ratio of formic acid and carbonyl reactant supplied to the catalyst is preferably in the range of from 0.2 to 5.0, more preferably of from 0.5 to 5.0. Even more preferably, the molar ratio is at least the stoichiometric ratio of 1.0. A molar ratio in the range of from 1.0 to 3.0 is particularly preferred.

It is an advantage of the process according to the invention that no molecular hydrogen is needed for the hydrogenation reaction. Preferably, the reactant is contacted with the heterogeneous catalyst in the absence of externally supplied molecular hydrogen, i.e. with formic acid as the sole hydrogen source. Molecular hydrogen that is generated upon dissociation of formic acid and not consumed by the hydrogenation reaction may be recycled to the catalyst. This in-situ formed molecular hydrogen is not considered as externally supplied molecular hydrogen.

The catalyst used in the process according to the invention is a heterogeneous catalyst comprising a hydrogenating compound. The hydrogenating compound preferably is a metal or a compound of a metal of any one of Columns 7 to 11 of the Periodic Table of Elements. Particularly suitable hydrogenating compounds are nickel, rhenium, copper or compounds thereof or noble metals such as platinum or palladium. Nickel or a nickel compound are particularly preferred.

The catalyst may comprise more than one hydrogenating compound or may comprise a further metal or metal compound as promoter. Any promoter known to be suitable for hydrogenation reactions may be used. Chromium or a chromium compound is an example of a suitable promoter.

The catalyst may comprise the hydrogenating compound on a solid catalyst carrier. Alternatively, the catalyst may comprise an unsupported hydrogenating compound. Examples of such unsupported catalysts are co-precipitated metal oxides, skeleton metal catalysts or fused metal catalysts.

Examples of suitable solid catalyst carriers are carbon or non-acidic refractory oxides such as neutralised alumina, titania, zirconia, clays or zeolites, or silica. Silica and carbon are particularly preferred catalyst carriers.

The hydrogenating compound is preferably at least partly in its metallic state under normal operating conditions. This may be achieved by reducing the catalyst prior to contacting it with the reactants. Reduction may also be achieved in-situ, i.e. during the hydrogenation process.

An advantage of the process according to the invention is that catalysts may be used that are much simpler than the homogeneous transition metal complexes used in the prior art processes.

The carbonyl reactant is contacted with the catalyst at a temperature in the range of from 150 to 400° C., preferably of from 200 to 350° C. The reaction may be carried out at ambient pressure. Elevated pressure, i.e. a pressure above ambient pressure may be applied in order to increase the conversion. Typically, the pressure will be in the range of from 1.0 to 150 bar (absolute), preferably of from 1.0 to 50 bar (absolute), more preferably of from 1.0 to 10.0 bar (absolute).

The process may be carried out as a continuous, a batch or a semi-batch process. If the process is carried out as a continuous process, the carbonyl reactant is preferably supplied to the catalyst at a weight hourly velocity in the range of from 0.1 to 10.0 kg/kg.hr (kilogram carbonyl reactant per kilogram catalyst per hour). If the process is a batch process, the residence time is preferably in the range of from 0.1 to 10.0 hr.kg/kg (hours times kilogram catalyst per kilogram carbonyl reactant).

### EXAMPLES

The invention will be further illustrated by means of the following non-limiting examples.

### Example 1

Catalyst particles and silicon carbide particles (catalyst:SiC weight ratio of 0.2) were loaded in a 5 mL quartz reactor tube with a length/diameter ratio of 10. The catalyst was then reduced under 10 wt % hydrogen in nitrogen at 300° C. for 16 hours.

The reactor tube was then maintained at 200° C. and a feed stream comprising undiluted formic acid and undiluted ethyl levulinate in a molar ratio of 1.0 was then continuously supplied to the catalyst at different weight hourly velocities. Reaction products were continuously withdrawn from the reactor tube and collected in an ice-cooled flask. The reactor was kept at ambient pressure. At these process conditions, reactants were in the gas phase when contacting the catalyst.

Different catalysts were used in four different experiments. The condensed reaction product was analysed by off-line gas chromatography analysis.

Experiment 1

A catalyst was used that was prepared by impregnating silica with a solution comprising nickel nitrate and Pt(NH₃)₄(NO₃)₂. The resultant catalyst particles comprised 10 wt % Ni and 0.05 wt % Pt.

Experiment 2

A catalyst was used that was prepared by impregnating silica with a solution comprising HReO₄ and Pt(NH₃)₄(NO₃)₂. The resultant catalyst comprised 10 wt % Re and 0.1 wt % Pt.

Experiment 3

A commercially available catalyst (1808T, ex. Engelhard) comprising co-precipitated Cu and Cr was used.

Experiment 4

A commercially available catalyst (UN-No2881; ex. Kataleuna) comprising Ni was used.

In Table 1, the catalyst used, the weight hourly velocity, ethyl levulinate (EL) conversion and yield of gamma valerolactone (gVL) based on moles of ethyl 10 levulinate in the feed stream, for the different experiments are shown.

TABLE 1
Experimental set-up and results for
EXAMPLE 1 (experiments 1 to 4)

| | | weight | | |
|---|---|---|---|---|
| | | hourly | | |
| | | velocity | EL | |
| | | (g EL/g | conversion | gVL yield |
| experiment | catalyst | cat · hr) | (% mole/mole) | (% mole/mole) |
| 1 | Ni/Pt on | 3.3 | 9.5 | 8.5 |
| | silica | 0.8 | 32 | 31 |
| 2 | Re/Pt on | 3.3 | 6.0 | 5.7 |
| | silica | 0.6 | 8.4 | 8.4 |
| 3 | co- | 7.0 | 8.3 | 7.2 |
| | precipitated | 1.5 | 12 | 11 |
| | Cu/Cr | | | |
| 4 | Ni | 5.0 | 40 | 38 |
| | | 1.5 | 74 | 71 |

### Example 2

A feed stream comprising undiluted formic acid and undiluted ethyl levulinate was contacted with a commercially available Ni-comprising solid hydrogenating catalyst (the same catalyst as used in experiment 4 of EXAMPLE 1) at different formic acid/ethyl levulinate molar ratios, different weight hourly velocities and different temperatures at ambient pressure. Reactor tube, catalyst dilution, catalyst pre-reduction and reactant supply and withdrawal were as described for EXAMPLE 1.

In Table 2 is shown the molar ratio of formic acid (FA) and ethyl levulinate (EL) in the feed stream, the operating temperature, weight hourly velocity of ethyl levulinate and gamma valerolactone (gVL) yield for the different experiments.

TABLE 2
Experimental set-up and yield for
EXAMPLE 2 (experiments 5 to 7)

| | | | weight hourly | |
|---|---|---|---|---|
| | | | velocity | gVL yield |
| | FA/EL | T | (g EL/g | (% of EL |
| experiment | (mole/mole) | (° C.) | cat · hr) | feed) |
| 5 | 1.0 | 200 | 5.0 | 40 |
| | | | 1.7 | 68 |
| 6 | 1.0 | 250 | 4.5 | 73 |
| | | | 0.9 | 84 |
| 7 | 3.0 | 250 | 1.1 | 81 |
| | | | 0.7 | 80 |

### Example 3

A feed stream comprising undiluted formic acid and undiluted levulinic acid at a molar ratio of 1.0 was contacted with a commercially available Ni-comprising solid hydrogenating catalyst (the same catalyst as used in experiment 4 of EXAMPLE 1 and in all experiments of EXAMPLE 2) at a weight hourly velocity of 3 g/g.hr (grams levulinic acid per gram catalyst per hour), at a temperature of 275° C. and at ambient pressure. Reactor tube, catalyst dilution, catalyst pre-reduction and reactant supply and withdrawal were as described for EXAMPLE 1.

A yield of 30 mole % gamma valerolactone and 7 mole % alpha-angelica lactone, based on the moles of levulinic acid in the feed stream, was obtained.

What is claimed is:

1. A hydrogenation process for the conversion of a reactant selected from the group consisting of a carboxylic acid having a carbonyl group and an ester of a carboxylic acid having a carbonyl group to form a carboxylic acid or an ester with a hydroxyl group or a lactone, wherein the reactant is contacted with a heterogeneous catalyst comprising a hydrogenating compound, in the presence of formic acid, at a temperature in the range of from 150 to 400° C. and a pressure in the range of from 1.0 to 150 bar (absolute).

2. A process according to claim 1, wherein the carbonyl group is a gamma- or a delta-carbonyl group and a gamma- or delta-lactone is formed.

3. A process according to claim 2, wherein the reactant is levulinic acid or an ester of levulinic acid.

4. A process according to claim 1, wherein the reactant is an ester.

5. A process according to claim 1, wherein formic acid and the reactant are supplied to the catalyst in a molar ratio in the range of from 0.2 to 5.0.

6. A process according to claim 1, wherein the reactant is contacted with the heterogeneous catalyst in the absence of externally supplied molecular hydrogen.

7. A process according to claim 1, wherein the hydrogenating compound is a metal or a compound of a metal of any one of Columns 7 to 11 of the Periodic Table of Elements.

8. A process according to claim 7, wherein at least part of the hydrogenating compound is in its metallic form.

9. A process according to claim 1, wherein the reactant is contacted with the catalyst at a temperature in the range of from 200 to 350° C.

10. A process according to claim 1, wherein the reactant is contacted with the catalyst at a pressure in the range of from 1.0 to 50 bar (absolute).

11. A process according to claim 1, wherein the reactant is an alkyl ester.

12. A process according to claim 1, wherein the reactant is a methyl, ethyl, butyl or pentyl ester.

13. A process according to claim 1, wherein formic acid and the reactant are supplied to the catalyst in a molar ratio in the range of from 1.0 to 3.0.

14. A process according to claim 1, wherein the hydrogenating compound is nickel or a nickel compound.

15. A process according to claim 14, wherein at least part of the hydrogenating compound is in its metallic form.

16. A process according to claim 10, wherein the reactant is contacted with the catalyst at a pressure in the range of from 1.0 to 10.0 bar (absolute).

17. A process according to claim 2, wherein the reactant is an ester.

18. A process according to claim 2, wherein the reactant is an alkyl ester.

19. A process according to claim 3, wherein the reactant is an ester.

20. A process according to claim 3, wherein the reactant is an alkyl ester.
